(19) United States
(12) Patent Application Publication
(10) Pub. No.: US 2024/0172989 A1
(43) Pub. Date: May 30, 2024

(54) NON-INVASIVE MEDICAL EXAMINATION USING ELECTRIC FIELDS

(71) Applicant: Zedsen Limited, London (GB)

(72) Inventor: Hrand Mami MAMIGONIANS, London (GB)

(21) Appl. No.: 18/510,247

(22) Filed: Nov. 15, 2023

(30) Foreign Application Priority Data
Nov. 30, 2022 (GB) 2217966.7

(51) Int. Cl. A61B 5/00; A61B 5/05; A61B 5/145

(52) U.S. Cl. CPC A61B 5/4312; A61B 5/05; A61B 5/14546; A61B 5/6805; A61B 5/7264; A61B 2560/0252

(57) ABSTRACT

Non-invasive medical examinations are performable in response to generated electric fields. Human tissue is located in contact with an apparatus having insulated electrodes mounted on a flexible dielectric membrane. A transmitting electrode is selected and a monitoring electrode is selected, such that electric fields penetrate the human tissue. The apparatus has a dielectric spacer with a first surface in contact with the dielectric membrane, a second surface, and a window between the first surface and the second surface. An infra-red sensor is located on the second surface and is configured to receive infra-red radiation from the flexible dielectric membrane, via the window, to determine the temperature of the flexible dielectric membrane. A processor is configured to produce output signals derived from the monitoring electrode that are compensated with reference to the determined temperature.

### CROSS REFERENCE TO RELATED APPLICATIONS

This application claims priority from United Kingdom Patent Application number 2217966.7, filed on Nov. 30, 2022, the whole contents of which are incorporated herein by reference.

### TECHNICAL FIELD

The present invention relates to the field of non-invasive medical examination and in particular to the use of electric fields in which electrodes are capacitively coupled.

### BACKGROUND OF THE INVENTION

It is known to examine body tissue using electric fields (as described in U.S. Pat. No. 11,484,244, assigned to the present applicant) for examining breast tissue. It is also known to use electric fields to examine the constituents of blood and, in particular, glucose concentrations in blood, as described in GB 2575718B and assigned to the present applicant. In both cases, the procedure involves bodily tissue coming into contact with capacitively coupled electrodes. This introduces a problem, in that it can no-longer be assumed that the electrodes, and substrate or membrane to which the electrodes are attached, will continue to operate at ambient temperature. Furthermore, experiments have shown that measurements can be influenced by temperature, which in turn can introduce errors.

Problems also exist in terms of obtaining a measurement of temperature that is consistent with temperature variations that introduce erroneous measurements. A known approach when examining a finger, for example, is to measure the actual temperature of the finger before and after deployment upon the insulated electrodes. Alternatively, temperature measuring devices may determine the temperature of air present within an examining apparatus. Again, experiment has indicated that neither of these approaches are reliable because they do not record the actual temperature of the components that are affected by temperature variations.

### BRIEF SUMMARY OF THE INVENTION

According to a first aspect of the present invention, there is provided an apparatus for performing non-invasive medical examinations in response to generated electric fields, comprising: a plurality of insulated electrodes mounted on a flexible dielectric membrane; a dielectric spacer having a first surface in contact with said dielectric membrane, a second surface, and a window between said first surface and said second surface; and an infra-red sensor located on said second surface and configured to receive infra-red radiation from said flexible dielectric membrane via said window to determine the temperature of said flexible dielectric membrane.

In an embodiment, the sides of said window defined by the dielectric spacer are angled to present a wider opening on said first surface, at the position of the membrane, compared to said second surface at the position of the infra-red sensor.

The non-invasive medical examinations may detect the concentration of one or more chemicals within circulating blood, in which the plurality of insulated electrodes are configured to be contacted by a finger; and the plurality of insulated electrodes are substantially linear and substantially parallel.

In an embodiment, the dielectric spacer includes a raised portion arranged to extend into an opening within an upper circuit board to support the dielectric membrane.

The non-invasive medical examination may detect anomalies in breast tissue, in which the flexible dielectric membrane is substantially dome-shaped, defining an internal surface arrange to be in contact with a human breast; the insulated electrodes comprise a first set of circular electrodes arranged in a configuration of concentric rings; and a second set of substantially radial electrodes overlap the concentric rings.

In an embodiment, an outer membrane is arranged over the substantially dome-shaped flexible dielectric membrane and the dielectric spacer may be positioned between the outer membrane and the substantially dome-shaped flexible dielectric membrane.

According to a second aspect of the invention, there is provided a method of performing non-invasive medical examinations, in response to generated electric fields, comprising the steps of: locating human tissue in contact with a plurality of insulated electrodes mounted on a flexible dielectric membrane; and selecting a transmitting electrode and a monitoring electrode from said plurality of insulated electrodes, such that electric fields penetrate said human tissue, wherein: a first surface of a dielectric spacer is in contact with the dielectric membrane; a window is provided between said first surface and a second surface of said dielectric spacer; an infra-red sensor is located on said second surface and is configured to receive infra-red radiation from the flexible dielectric membrane via said window, to determine the temperature of the flexible dielectric membrane, and further comprising the steps of: producing output signals derived from the monitored electrode; and compensating said output signals with reference to said determined temperature.

Embodiments of the invention will be described, by way of example only, with reference to the accompanying drawings. The detailed embodiments show the best mode known to the inventor and provide support for the invention as claimed. However, they are only exemplary and should not be used to interpret or limit the scope of the claims. Their purpose is to provide a teaching to those skilled in the art. Components and processes distinguished by ordinal phrases such as “first” and “second” do not necessarily define an order or ranking of any sort.

### BRIEF DESCRIPTION OF THE SEVERAL VIEWS OF THE DRAWINGS

FIG. 1 shows an apparatus for performing non-invasive medical examinations;

FIG. 2 details internal components of the apparatus shown in FIG. 1;

FIG. 3 shows the underside of a main circuit board identified in FIG. 2;

FIG. 4 shows a schematic representation of the apparatus shown in FIG. 1;

FIG. 5 illustrates the operation of the apparatus shown in FIG. 1;

FIG. 6 shows further operation of the apparatus shown in FIG. 1;

FIG. 7 shows further operation of the apparatus shown in FIG. 1;

FIG. 8 shows the apparatus of FIG. 1 displaying output information;

FIG. 9 shows a cross-section of the apparatus shown in FIG. 1;

FIG. 10 shows a top view of an acetyl dielectric spacer identified in FIG. 9;

FIG. 11 shows an alternative embodiment presented as a case for a conventional mobile (cellular) telephone;

FIG. 12 shows output data being displayed on the apparatus identified in FIG. 11;

FIG. 13 shows a second alternative embodiment, in the form of a wearable item;

FIG. 14 shows an exploded view of the wearable item identified FIG. 13;

FIG. 15 shows a cross-section of a dome-shaped substrate identified in FIG. 14;

FIG. 16 shows a silicone rubber dielectric spacer identified in FIG. 15;

FIG. 17 illustrates the deployment of radial electrodes in the embodiment of FIG. 13;

FIG. 18 shows the use of polar coordinates for identifying regions;

FIG. 19 shows a plan view of an inner surface of the flexible substrate identified in FIG. 15;

FIG. 20 shows a plan view of the outer surface of the flexible substrate identified in FIG. 15;

FIG. 21 illustrates a process of machine learning;

FIG. 22 details a machine learning procedure identified in FIG. 21;

FIG. 23 shows an example of a procedure for energizing electrodes as identified in FIG. 22; and

FIG. 24 shows further procedures for energizing electrodes identified in FIG. 22.

### DETAILED DESCRIPTION OF EMBODIMENTS OF THE INVENTION

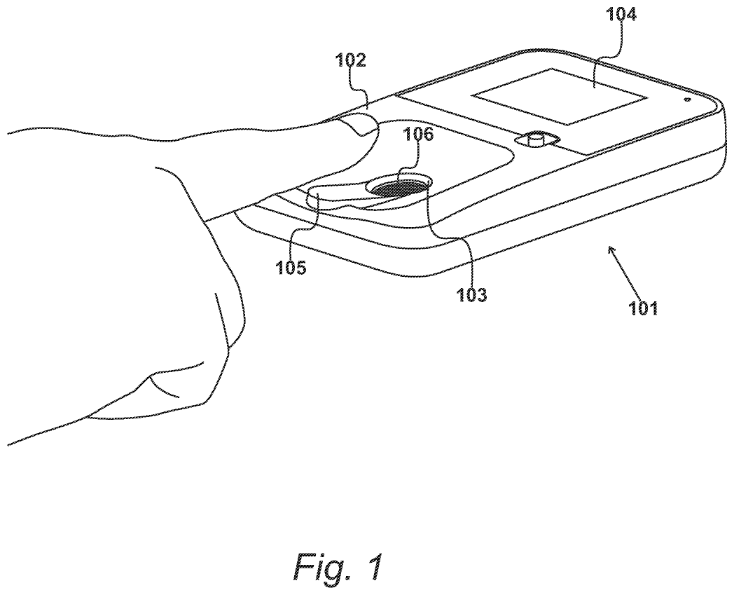

### FIG. 1

An apparatus 101 for performing non-invasive medical examinations in response to generated electric fields is shown in FIG. 1. The apparatus has a plastic housing 102 with a membrane-exposing orifice 103 presenting an application region arranged to make skin contact. In this embodiment, contact is made against the skin of a finger but in other embodiments, contact could be made with other suitable areas of the body.

The apparatus includes a visual display orifice 104 which, in this embodiment, is covered by a transparent cover, thereby allowing a visual display unit, supported by a main circuit board, to be seen during the operation of the apparatus. In this embodiment, the visual display unit is a liquid crystal display but other types of display could be deployed. In alternative embodiments, the display could take the form of devices configured to emit light of various colours. Alternatively, a display could be presented to a subject (the person being tested) or to an operative, such as a clinician, via an alternative device, such as a wireless-connected mobile device.

A guide portion 105 guides a subject's finger into position, to contact with an electrode supporting membrane 106.

In this embodiment, the device also measures applied force, thereby allowing a processor to compare force data against a predetermined level. Experiment has shown that a minimum level of pressure is required to ensure that reliable contact is made between the subject's finger and the electrode-supporting flexible dielectric membrane 106. In this embodiment, testing is inhibited if the assessed force data is not above this predetermined level.

A plurality of insulated electrodes are mounted on the flexible dielectric membrane 106, which is itself supported above the membrane-exposing orifice 103. In this way, when pressure is applied to the membrane, a limited degree of movement is possible, resulting in force being applied to a force sensor.

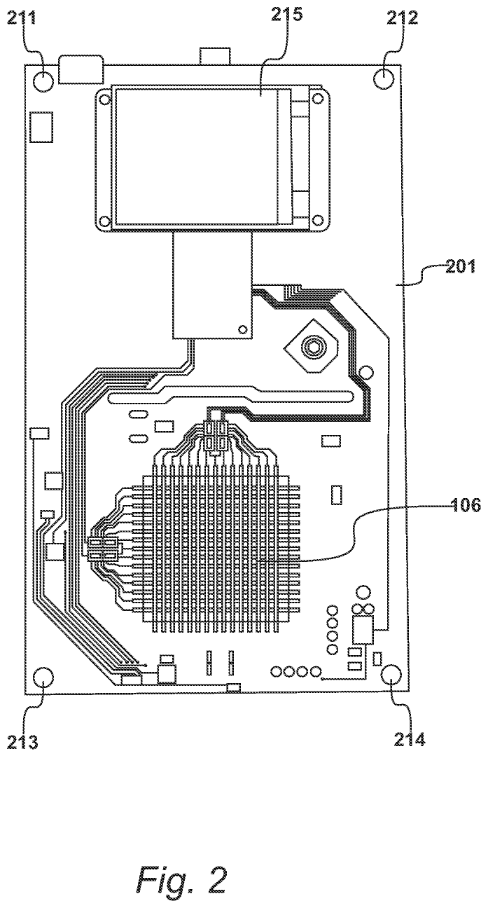

### FIG. 2

In an embodiment, the non-invasive medical examinations detect the concentration of one or more chemicals within circulating blood and, in an embodiment, concentrations of glucose are measured.

In the embodiment of FIG. 2, the plurality of insulated electrodes are configured to be contacted by a finger and are substantially linear and substantially parallel. This embodiment also includes additional insulated electrodes that are also substantially linear and parallel but are mounted on the opposite side of the flexible dielectric membrane and are substantially orthogonal to the first set of electrodes. The insulated electrodes are mounted on the dielectric membrane 106 and are connected to a main circuit board 201.

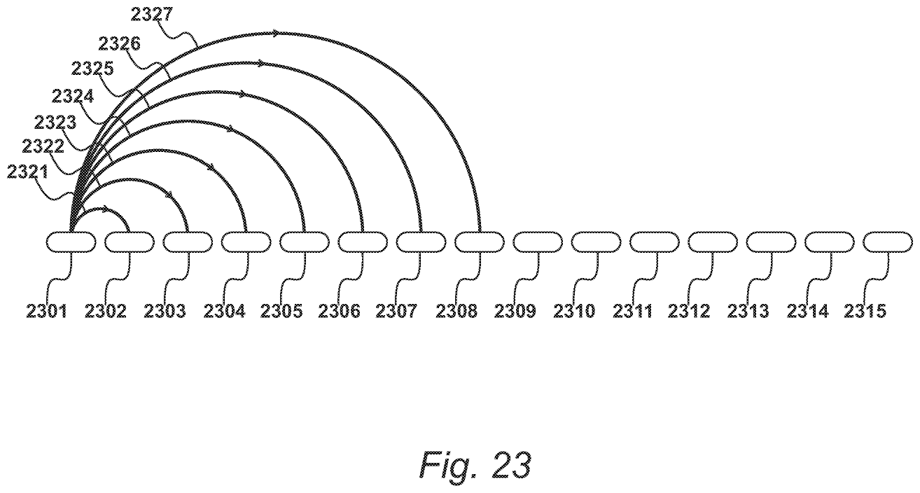

In an embodiment, a first layering operation (as described with reference to FIG. 23) is performed using the first group of insulated electrodes, followed by a second layering operation performed using the second group. In addition, conventional position detection is also possible, by sequentially energizing electrodes of one of these groups while monitoring selected electrodes of the other group.

In the embodiment of FIG. 2, the main circuit board 201 is secured to the housing 102 at a first securing location 211, a second securing location 212, a third securing location 213 and a fourth securing location 214. A visual display unit 215 is attached to the main circuit board 201.

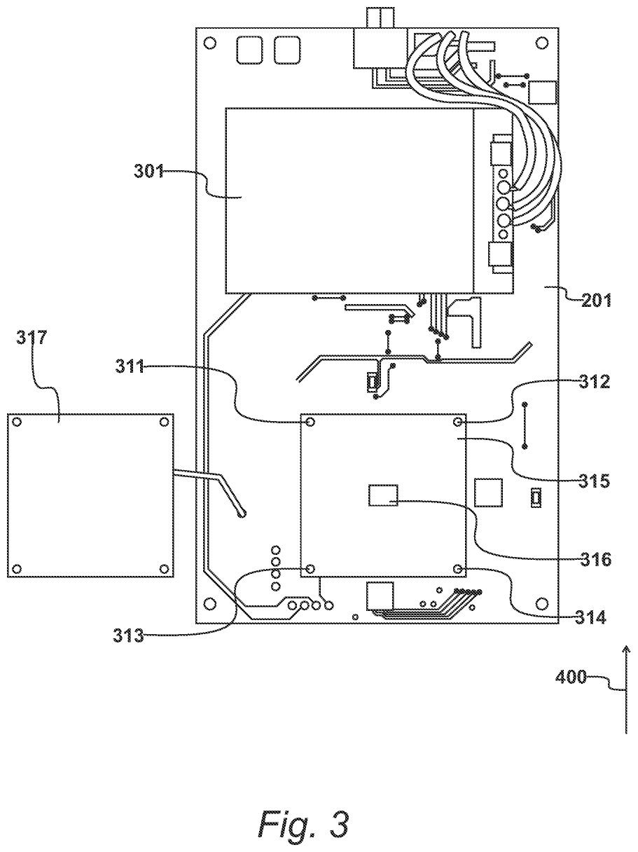

### FIG. 3

The underside of the main circuit board 201 is shown in FIG. 3. A rechargeable battery 301 provides electrical power for components within the apparatus. Fixing elements are provided by a first rod 311, a second rod 312, a third rod 313 and a fourth rod 314 which are secured to the main circuit board 201. An acetyl support 315 is located on rods 311 to 314 to support the dielectric membrane 106. In this embodiment, an acetyl material has been selected because the electrical properties of this material do not change with respect to changes in temperature and humidity experienced within the operational environment. The acetyl material therefore provides a dielectric spacer having a first surface in contact with the dielectric membrane in addition to a second surface.

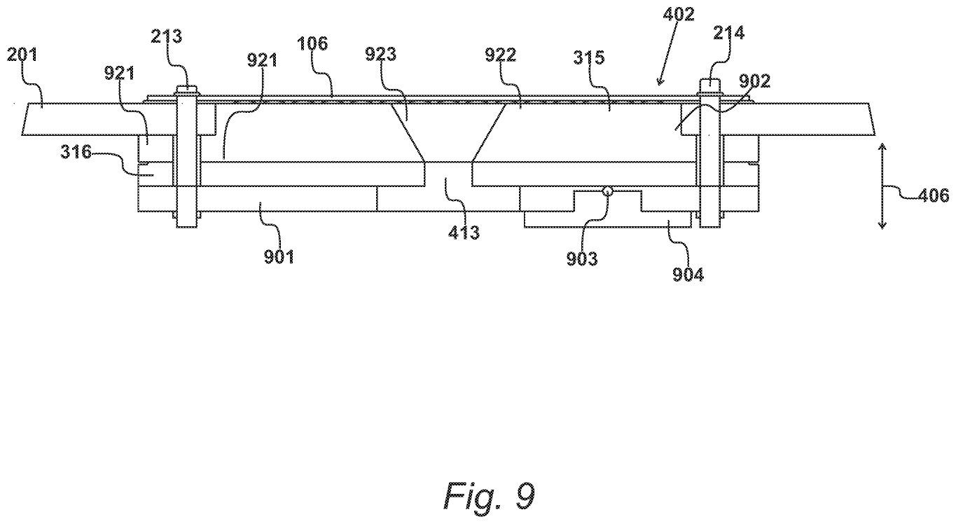

As described with reference to FIG. 9, a window 316 is provided between the first surface and the second surface of this dielectric spacer 315. Furthermore, an infrared sensor is located on the second surface that is configured to receive infrared radiation from the flexible dielectric membrane via the window to determine the temperature of the flexible dielectric membrane.

Following the application of the acetyl support 315 and the infrared sensor, an intermediate board 317 is deployed over the rods 311 to 314, such that this intermediate board 317 is guided, but not restrained, by these fixing elements. In this way, the intermediate board 317 is allowed to move, which results in the application of force onto a force sensor. In the embodiment of FIG. 3, the intermediate board 317 includes an electrically conductive ground plane to provide electrical shielding for the lower side of the membrane 106.

After deploying the intermediate board 317, a bottom circuit board is located on the fixing elements 311 to 314 and thereafter secured to the fixing elements. Thus, the fixing elements secure the bottom circuit board to the top circuit board, such that the bottom circuit board does not move with respect to the main circuit board and the bottom circuit board does not contact the housing 102 directly.

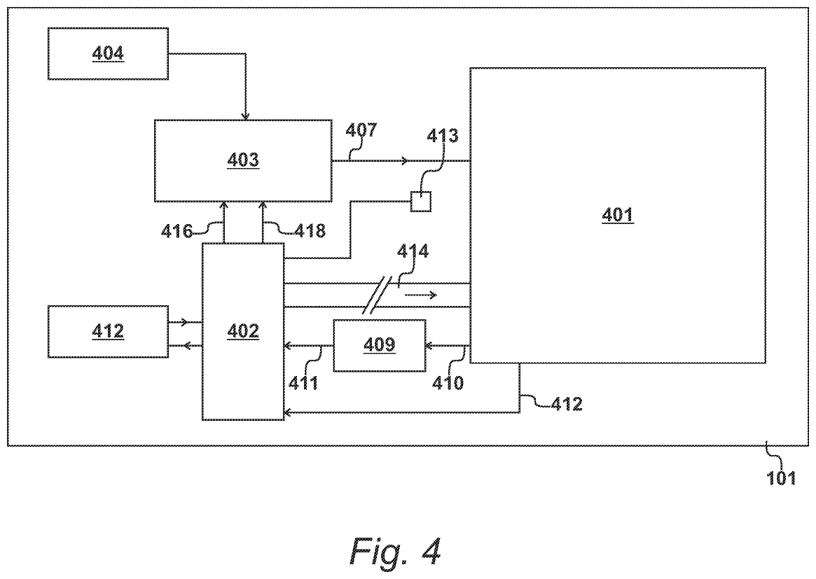

### FIG. 4

A schematic representation of the apparatus 101 is shown in FIG. 4. A multiplexing environment 401 includes the dielectric membrane supporting the insulated electrodes, along with a demultiplexer for demultiplexing energizing input pulses and a multiplexer for combining selected output signals. A processor 402 addresses the demultiplexer and the multiplexer to ensure that the same electrode cannot be both energized as a transmitter and monitored as a receiver during the same coupling operation. An energizing circuit 403 is energized by a power supply 404 and the energizing circuit 403 supplies energizing input signals to the multiplexing environment over an input line 407.

An output from the multiplexing environment is supplied to an output circuit 409 over a first analog line 410. A conditioning operation is performed by the output circuit 409, allowing analog output signals to be supplied to the processor 402 via a second analog line 411. The processor 402 also communicates with a data communication circuit 412 to facilitate communication with an attached mobile device using a wireless protocol.

During scanning operations, the processor 402 supplies addresses over address buses 414 to the multiplexing environment 401, to define a pair of capacitively coupled electrodes. An energization operation is performed by applying an energizing voltage, monitoring a resulting output signal and sampling the output signal a multiple number of times to capture data indicative of a peak value and a rate of decay.

The generation of an input impulse signal is controlled by the processor 402 by the generation of a control signal on a control line 416. The transition time required may be pre-programmed into the processor 402 and this information is conveyed to the energizing circuit via a control bus 418.

A data bus 421 provides data from the multiplexing environment 401 to the processor 402, representing applied pressure. An infrared sensor 413 provides data to the processor 402, identifying the temperature of the flexible dielectric membrane. Thus, in this way, it is possible to directly determine the temperature of the flexible dielectric membrane as described with reference to FIG. 9. In some embodiments, additional data may be provided to the processor 402, such as the temperature and humidity within the housing 102.

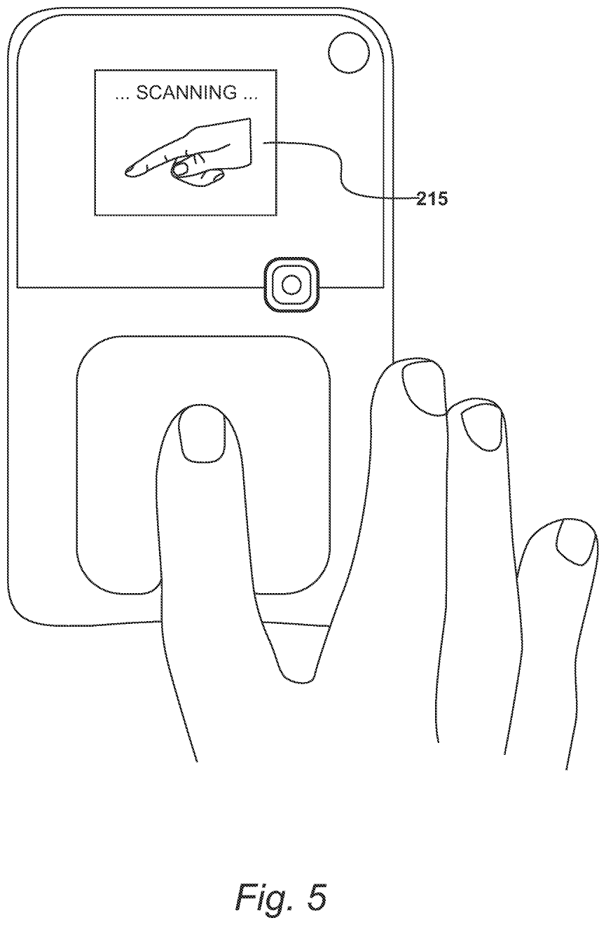

### FIG. 5

In operation, the visual display unit 215 invites a subject to deploy a finger within the guide portion 105 to engage with the membrane 106. The insulated electrodes are supported by the main circuit board and are exposed through the first membrane-exposing orifice of the housing.

The processor 404 energizes and monitors selected electrodes to produce output data which, in the embodiment of FIG. 5, indicates concentrations of blood glucose. The visual display unit 215 confirms this operation by indicating that the apparatus is scanning. Furthermore, throughout this procedure, the applied pressure and temperature of the membrane are monitored.

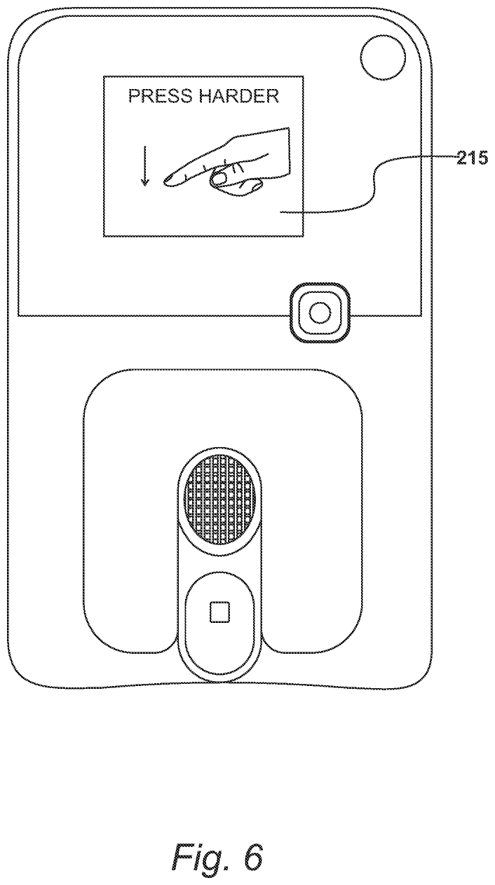

### FIG. 6

It is possible for the apparatus to determine that an insufficient pressure has been applied. When such a situation occurs, an invitation is generated as illustrated in FIG. 6. Thus, in response to an evaluation being made to the effect that the applied pressure is too low, the subject, via the visual display unit 215, is invited to press harder on the detector, so that the procedures may be repeated and reliable results obtained.

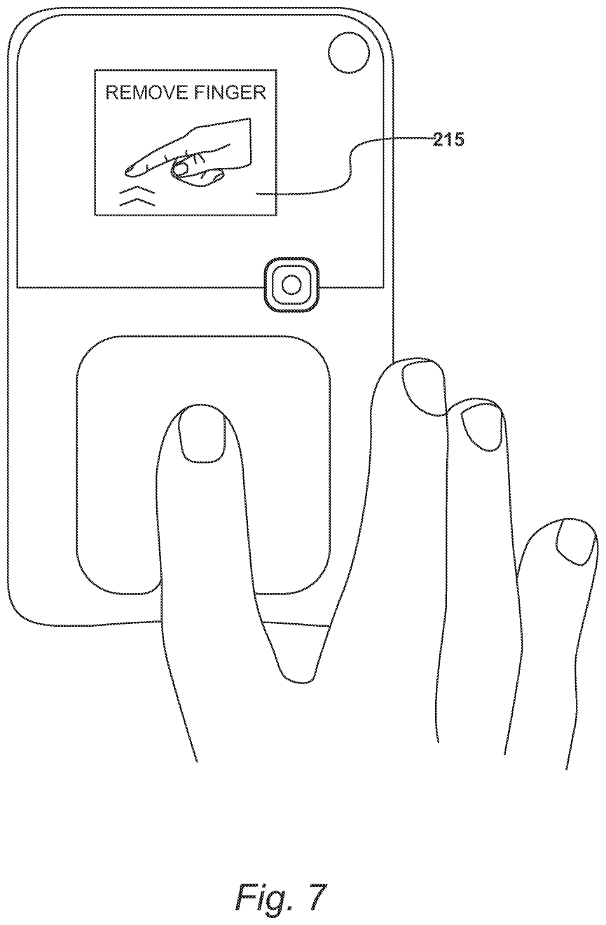

### FIG. 7

After the measuring process has completed, the visual display unit 215 invites the subject to remove their finger, as shown in FIG. 7.

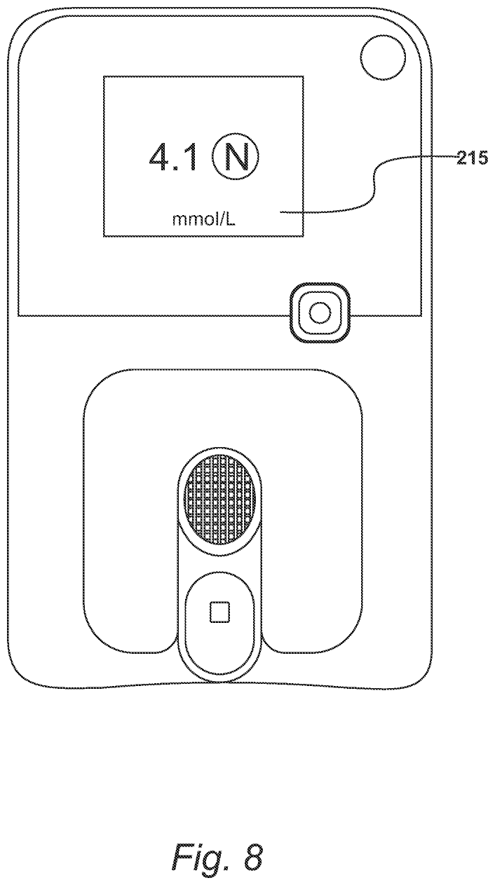

### FIG. 8

After analysing output data received during the scanning procedure, the visual display unit may provide an indication of concentrations, such as glucose concentration, as illustrated in FIG. 8. Furthermore, in addition to providing a numerical value, an indication may also be provided as to whether this concentration is considered to be too low, normal or too high. In an embodiment, for each of these possibilities, an appropriate colour is displayed.

### FIG. 9

Metal rod 213 and metal rod 214 are shown in FIG. 9 and these, along with the other two fixing rods 211, 212, secure a bottom circuit board 901 to the main circuit board 201. The main circuit board 201 includes a first orifice 902 and the dielectric membrane 106 is supported over this orifice. In an embodiment, the membrane has a thickness of typically 0.1 mm and the main circuit board 201 has a typical thickness of 1.6 mm. Each of the metal rods has an upper end and a lower end such that, in the embodiment, the upper ends are soldered to the main circuit board 201 and the lower ends are soldered to the bottom circuit board 901.

The dielectric spacer 315 is shown in FIG. 9, along with an intermediate board 316 having a ground plane. The dielectric spacer 315 and the intermediate board are guided by the fixing elements but are not restrained by these fixing elements, such that they are free to move in a vertical direction. Relatively little movement may occur, typically up to a maximum of ten micro-metres and it is unlikely that this would be perceived by a subject. Movement is restrained by a metal ball 903 extending from a force sensor 904, where the metal ball is in contact with the ground plane attached to the intermediate board 316.

The force sensor 904 is received within an orifice provided within the bottom circuit board 901, with the metal ball extending above the plane of the bottom circuit board 901. Thus, in this way, an extending portion of the force sensor extends above the top surface of the bottom circuit board 901. In an embodiment, the extending portion is surrounded by an elastomeric material which may be a silicone rubber having a shore durometer (type A) of less than forty. Thus, when flexing occurs, due to applied pressure, the elastomeric material compresses. Thereafter, when force is removed, the elastomeric material will expand back to its original position, thereby ensuring that the apparatus is returned to a fully operational state.

The dielectric spacer 315 is in contact with a second surface of the main circuit board as shown at 921. In an embodiment, the dielectric spacer has a raised portion 922 which engages within the first orifice 902 of the main circuit board to support the dielectric membrane. The raised portion includes a window 923 configured to allow infrared radiation from the membrane to be received by the infrared sensor 413 to provide a direct measurement of the temperature of the dielectric membrane.

In the embodiment shown in FIG. 9, the acetyl dielectric spacer 315 is in contact with an intermediate board and the infrared sensor 413 is located in this board. In this embodiment, the sides of the window defined by the dielectric spacer are angled to present a wider opening at the first surface, at the position of the membrane, compared to the opening at the second surface at the position of the infrared sensor or 413. Thus, in the embodiment of FIG. 9, the infrared sensor is located on an intermediate circuit board, the intermediate circuit board is in contact with a force sensor and the processor is configured to inhibit an examination procedure if an applied force is below a predetermined threshold.

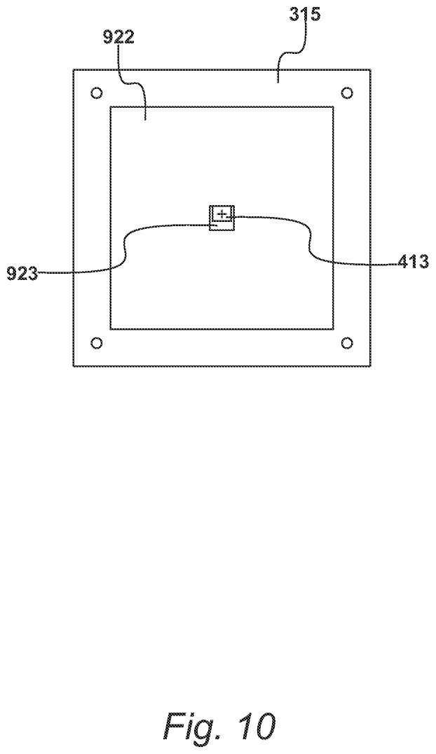

### FIG. 10

A top view of the acetyl dielectric spacer 315 is shown in FIG. 10. The spacer includes the raised portion 922 and, in FIG. 10, the top opening of the window 923 is shown. Furthermore, the infrared sensor 413 can be seen at the bottom of the window 923.

The infrared sensor 924 may be implemented as an MLX90632 device available from Melexis. The device is identified as a far infrared, non-contact temperature sensor, with high-accuracy factory calibration. Internally, electrical and thermal precautions are taken to compensate for ambient conditions. A sensing element produces a voltage signal that is amplified and digitized. After digital filtering, the measurement result is stored in random access memory. In addition, the device contains a sensor element to measure the temperature of the sensor itself. Again, this information is available from internal memory after being processed. The results of each measurement conversion are accessible via an I²C interface.

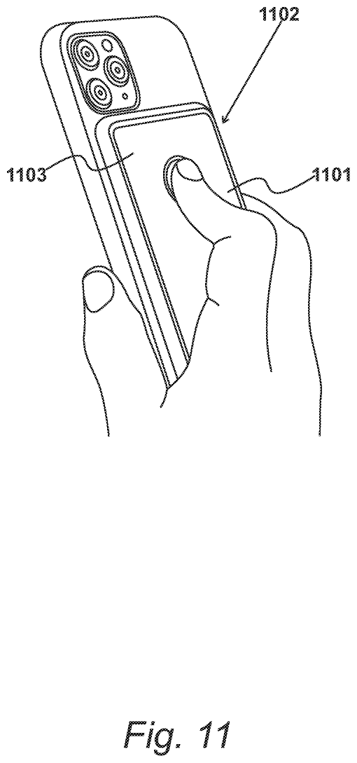

### FIG. 11

An alternative apparatus for performing non-invasive medical examinations in response to generated electric fields is shown in FIG. 11. As an alternative to being a stand-alone device, the apparatus 1101 is presented as a case for a conventional mobile (cellular) telephone 1102.

A housing 1103 is attached to the mobile (cellular) telephone that facilitates an interaction with a finger placed upon an appropriate electrode-supporting membrane, which is itself supported by a dielectric spacer having a first surface in contact with a dielectric membrane, a second surface and a window between the first surface and the second surface. Thus, in this way, in a manner substantially similar to that described with reference to FIG. 9, an infrared sensor is located on the second surface and is configured to receive infrared radiation from the flexible dielectric membrane via the window to determine the temperature of the flexible dielectric membrane.

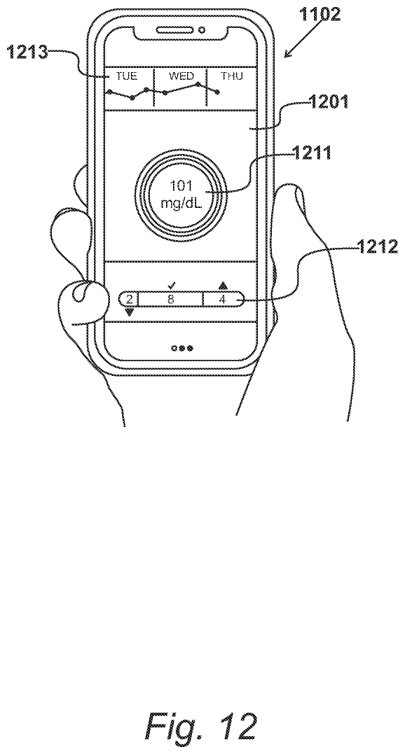

### FIG. 12

As illustrated in FIG. 12, in this alternative embodiment, a touch sensitive visual display 1201 of the mobile (cellular) telephone 1102 is used to facilitate a subject's interaction with the apparatus. In an embodiment, an appropriate software application is installed upon the mobile (cellular) telephone, thereby allowing a subject to initiate a medical examination; possibly, in this embodiment, involving the measurement of glucose concentration in the subject's blood. Thus, having performed an examination and obtained relevant data, graphical areas allow this information to be visually displayed to the subject.

In the embodiment shown in FIG. 12, the graphical user interface includes a first element 1211 which shows the results of a current examination taken substantially in a manner similar to that described with reference to FIG. 7 and FIG. 8.

In this embodiment, a second element 1212 indicates whether the measurement is considered low, normal or high and this in turn may prompt a subject to take appropriate medical intervention. In addition, in this embodiment, a third element 1213 displays historical data, thereby allowing trends to be considered.

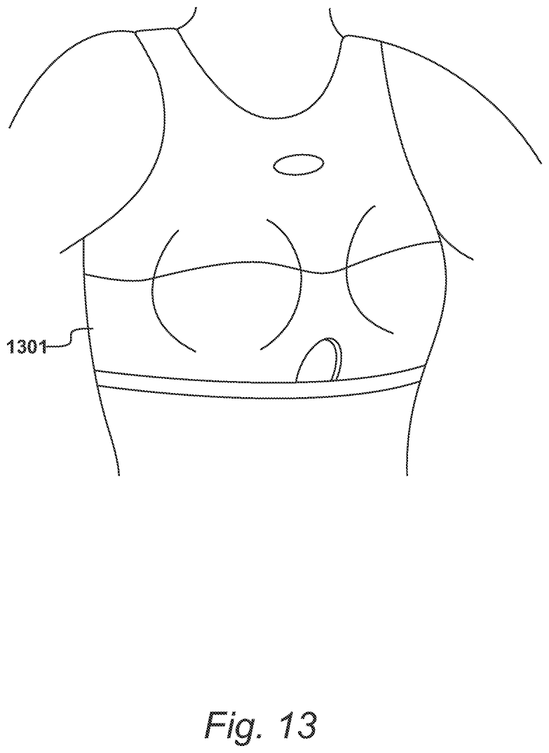

### FIG. 13

A second alternative embodiment of an apparatus for performing non-invasive medical examinations in response to generated electric fields will be described with reference to FIG. 13 to FIG. 20. The apparatus is included within a wearable item 1301 as shown in FIG. 13, configured to identify irregularities in breast tissue. A plurality of insulated electrodes are mounted on a flexible dielectric membrane and the apparatus includes an energizing circuit for energizing a selected transmitter electrode to generate an electric field, along with a monitoring circuit for receiving output signals from a selected receiver electrode, in a manner substantially similar to that described with reference to FIG. 4.

In this embodiment, the flexible substrate is substantially dome-shaped and defines an internal surface arranged to accommodate breast tissue. In an embodiment, it is possible for the apparatus to be deployed over a first breast and then over a second breast. However, in the embodiment of FIG. 13, a first flexible substrate is supported within the wearable item 1301, along with a second flexible substrate that is also supported within the wearable item.

The embodiment of FIG. 13 is designed to be deployed by a subject themselves and is configured to generate an alert signal if irregularities are detected. A subject is therefore in a position to conduct an examination on a regular basis and seek further advice should any irregularities be detected. The insulated nature of the electrodes ensures that they are electrically insulated from contacting tissue.

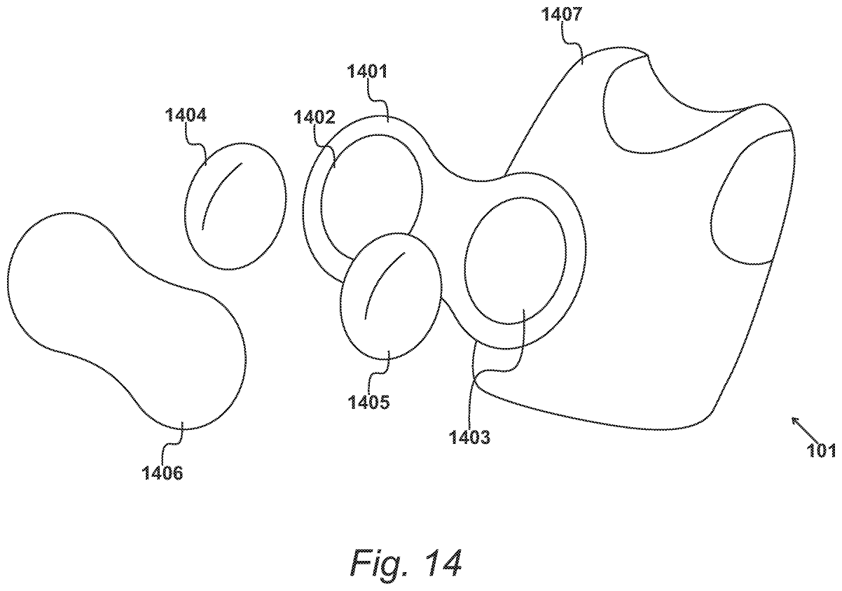

### FIG. 14

An exploded view of the wearable item 1301 is shown in FIG. 14, illustrating how the wearable item 1301 may be assembled to support the apparatus. The apparatus is supported on a frame 1401, defining a first opening 1402 and a second opening 1403, configured to receive a right dome-shaped substrate 1404 and a left dome-shaped substrate 1405 respectively. The right dome-shaped substrate 1404 and the left dome-shaped substrate 1405 are then covered by an outer cover 1406 which is then attached to a main support vest 1407.

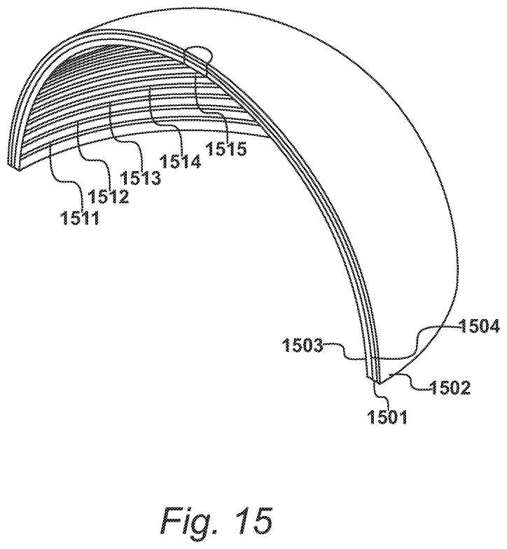

### FIG. 15

A cross-section of the right dome-shaped substrate 1404 is shown in FIG. 15. The substrate 1404 is constructed from an insulating dielectric material which may include a knitted or woven fabric, thereby allowing the shape of the substrate to adapt to the contours of the underlying biology. In this way, the apparatus remains comfortable, while ensuring that the capacitively coupled electrodes maintain an optimized relationship with respect to the tissue for which an attempt is being made to identify irregularities.

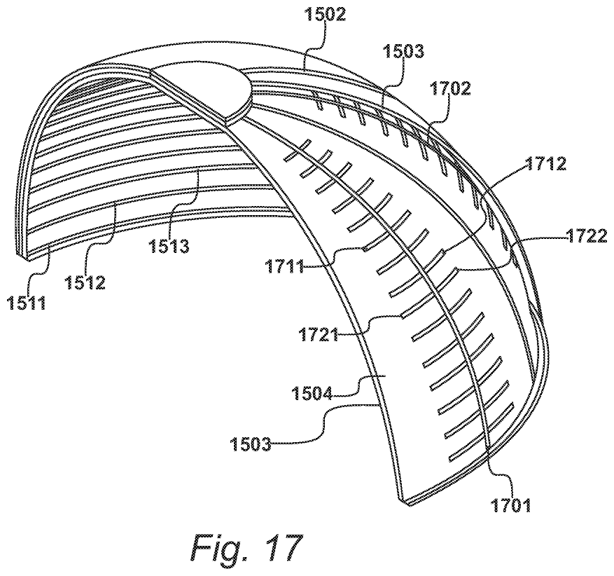

In the embodiment of FIG. 15, a dielectric spacer, providing support for the flexible substrate, is implemented as a silicone rubber shell 1501. A grounding cover 1502 provides shielding to prevent external electric fields from inducing noise. As shown in FIG. 15 and FIG. 17, the geometry of the plurality of insulated electrodes is significantly different from the embodiments previously described. However, in a manner substantially similar to that described with reference to FIG. 4, an energizing circuit energizes a selected transmitter electrode to generate an electric field and a monitoring circuit receives and samples output signals to provide output data. This output data may be retained locally or supplied to other external equipment.

The electrode geometry is such as to evenly arrange the electrodes over the dome-shaped substrate. In the embodiment of FIG. 15, an arrangement of electrodes has been achieved by providing circular electrodes that are arranged as concentric rings that, in the embodiment of FIG. 15, are located on an inner surface 1503 of the substrate 1404. These are identified as a first set of electrodes and include a first circular electrode 1511, a second circular electrode 1512, a third circular electrode 1513, a fourth circular electrode 1514 and a fifth circular electrode 1515. In an alternative embodiment, this first set of electrodes could be located on an outer surface of the flexible dielectric membrane.

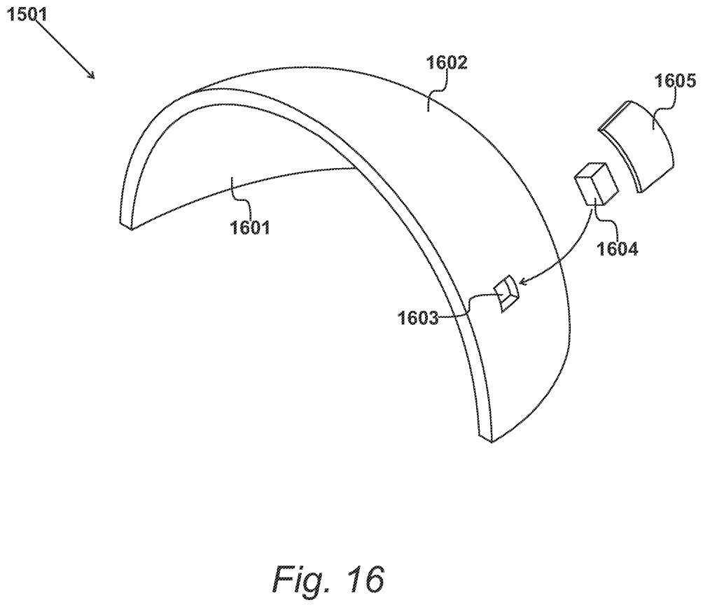

### FIG. 16

The silicone rubber dielectric spacer 1501 is detailed in FIG. 16. The dielectric spacer 1501 has a first surface 1601 in contact with the dielectric membrane and a second surface 1602. A window 1603 is provided between the first surface 1601 and the second surface 1602. An infrared sensor 1604 is located on the second surface 1602 and is configured to receive infrared radiation from the flexible dielectric membrane via the window 1603 to determine the temperature of the flexible dielectric membrane. The infrared sensor 1604 is held in place by a cover 1605.

A single infrared sensor 1604 is shown in FIG. 16 but in an embodiment, a plurality of devices of this type are included and an average temperature value may be determined from the resulting plural outputs.

### FIG. 17

In an embodiment, as illustrated in FIG. 17, radial electrodes are included in addition to the circular electrodes described with reference to FIG. 15. The radial electrodes have been positioned on the outer surface of the dome-shaped flexible substrate; and in FIG. 17, a portion of the grounding cover 1502 and a portion of the silicone shell 1503 have been removed to reveal a first radial electrode 1701 and a second radial electrode 1702.

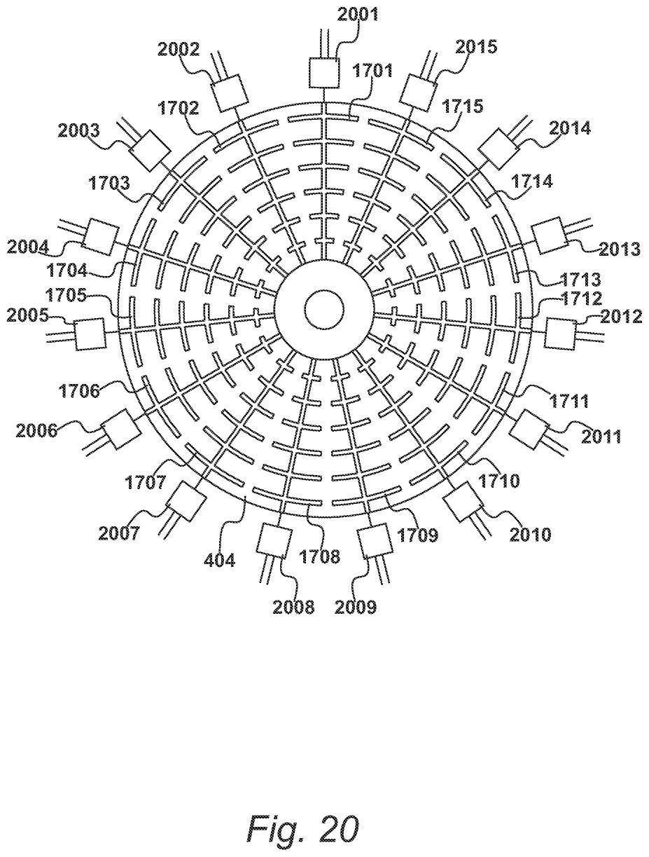

In this embodiment, each radial electrode, such as the first radial electrode 1701, includes first branches 1711 extending from a first side, along with second branches 1712 extending from a second side. Each branch defines a first tip 1721 on the first side, with a similar second tip 1722 being defined on the second side. In this embodiment, distances between adjacent tips of adjacent branches are substantially similar, as described with reference to FIG. 20.

Thus, this embodiment provides a flexible dielectric membrane that is substantially dome-shaped, defining an internal surface arranged to be contacted with a human breast. The insulated electrodes comprise a first set of circular electrodes arranged in a configuration of concentric rings, along with a second set of substantially radial electrodes that overlap the concentric rings. Furthermore, as described with reference to FIG. 16, the dielectric spacer is positioned between the outer membrane and the substantially dome-shaped flexible dielectric membrane.

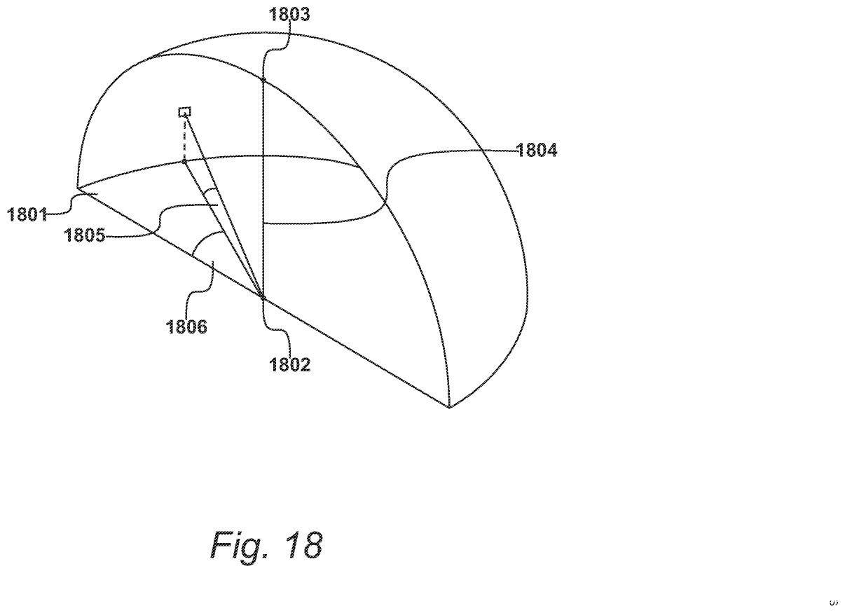

### FIG. 18

To identify locations within breast tissue, the embodiment described with reference to FIG. 15 to FIG. 17 models the region as a hemisphere, with locations specified by polar coordinates as illustrated in FIG. 18. In the geometric model of FIG. 18, an XY plane 1801 is defined having a centre or origin 1802. The origin 1802 lies directly below a centre point 1803 of the hemispherical dome.

Within this model, a radius 1804 remains constant but, in alternative embodiments, it is possible for variations to occur, thereby allowing the model to adopt to more natural irregular dome-shaped configurations.

The circular electrodes 1511 to 1515 have positions that may be identified by a latitude angle 1805. Similarly, the positions of the radial electrodes 1701, 1702 etc may be identified by a longitude angle 1806. Thus, any position within the region of the tissue may be defined by appropriate polar coordinates.

The radial electrodes also divide the hemisphere into a plurality of segments. Consequently, comparisons may be made between segments that have substantially similar positions in relation to the right breast and the left breast. In healthy tissue, resulting measurements from these two segments should be substantially similar. However, if a significant difference is identified, this may suggest that an irregularity exists within one of the measured segments. Thus, by adopting this technique, it is possible to identify an irregularity without making reference to historical data and without making reference to external databases.

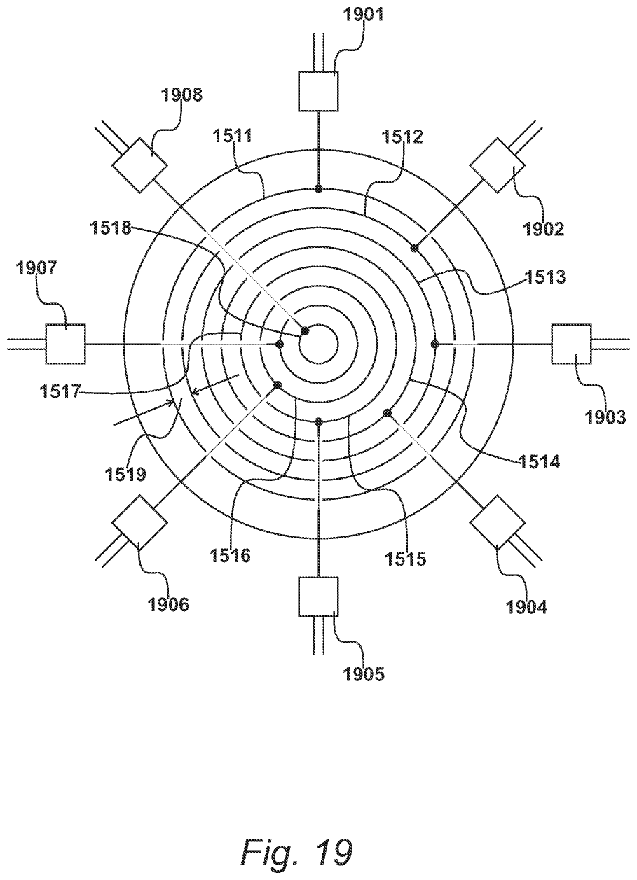

### FIG. 19

A plan view of the inner surface of the flexible substrate is illustrated in FIG. 19, which shows the first circular electrode 1511 to the fifth circular electrode 1515, along with a sixth circular electrode 1516, a seventh circular electrode 1517 and an eighth circular electrode 1518. In this embodiment, a distance 1519 between adjacent circular electrodes is substantially constant. The object is to provide similar output results at different locations for the tissue being examined.

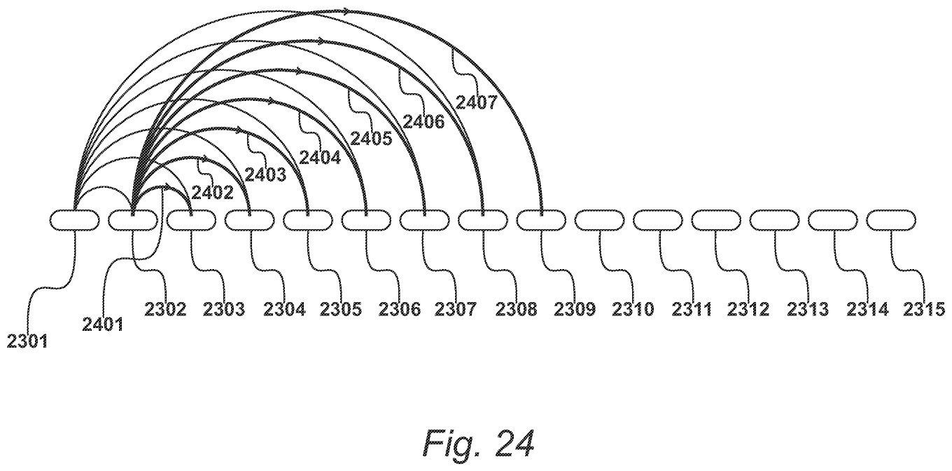

In the embodiment shown in FIG. 19, a first set of electrodes are present on the inner surface and a second set of electrodes are present on the outer surface. Separate multiplexing means are provided for each set of electrodes, such that each respective multiplexing means is configured to select any electrode of its respective set to be a transmitter electrode while selecting any of the remaining electrodes in the set to be a receiver electrode. This allows sophisticated scanning techniques to be deployed as described with reference to FIG. 23 and FIG. 24.

In this embodiment, a first multiplexing device 1901 is attached to the first circular electrode, with a similar second multiplexing device 1902 being connected to the second circular electrode and so on until an eighth multiplexing device 1908 is attached to the eighth circular electrode 1518.

### FIG. 20

A plan view of the outer surface of the flexible substrate is illustrated in FIG. 20, showing the radial electrodes, including the first radial electrode 1701 and the second radial electrode 1702, along with a third radial electrode 1703 and so on to a fifteenth radial electrode 1715. A respective multiplexing device is provided for each radial electrode, consisting of a first radial multiplexing device 2001 to a fifteenth radial multiplexing device 2015. Thus, again, this allows any of the electrodes to be selected as a transmitter electrode, with any of the remaining electrodes being selected as a receiver electrode.

The arrangement of the first set of electrodes and the second set of electrodes allows specific regions of the tissue to be identified and examined. As described with reference to FIG. 16, the presence of the infrared sensors allows temperature evaluations to be made of the membrane itself and appropriate compensation included, thereby allowing accurate comparisons to be made for examinations carried out at different temperatures. In this way, it is possible for accurate comparisons to be made with respect to historical data and data derived from external sources.

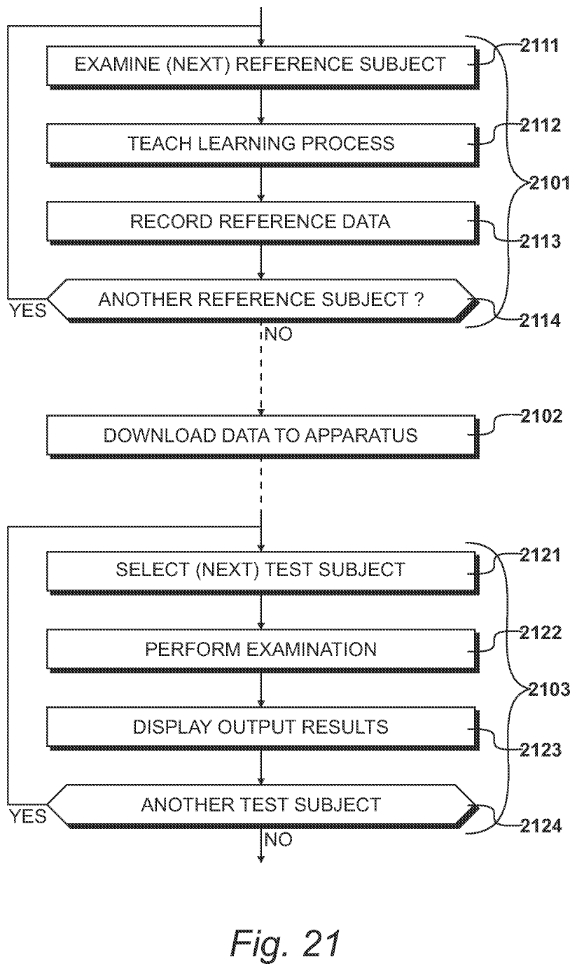

### FIG. 21

The embodiments described with reference to FIG. 1 to FIG. 20 facilitate the deployment of a method of performing non-invasive medical examinations in response to generated electric fields. Human tissue is located in contact with a plurality of insulated electrodes mounted on a flexible dielectric membrane. A transmitter electrode is selected from the plurality of insulated electrodes and a second electrode is monitored, such that electric fields penetrate the human tissue. A dielectric spacer is provided having a first surface in contact with the dielectric membrane, along with a second surface. A window is provided between the first surface and the second surface which allows an infrared sensor to be located on the second surface and is configured to receive infrared radiation from the flexible dielectric membrane via the window to determine the temperature of the flexible dielectric membrane. Furthermore, a processor is configured to produce output signals derived from the monitoring electrodes that are compensated for the determined temperature.

In an embodiment, following experiment and analysis, it is possible to deploy a model which specifies how measurements taken from monitored electrodes are influenced with respect to changes in temperature. However, in the embodiment illustrated in FIG. 21, instructions are developed and reference data is developed for operations performed by the processor to facilitate the production of output signals by a process of machine learning. Thus, preproduction steps 2101 are identified in FIG. 21.

Having produced this reference data, a production procedure includes the downloading of data to the apparatus at step 2102. Within the apparatus itself, this data may be retained in non-volatile memory and, in an embodiment, the combination may be identified as firmware; in that, this data may be updated remotely after further iterations of steps 2101.

In an embodiment, the process of machine learning evaluates many examinations in which tissue characteristics are known and the temperature of the evaluating membrane is also known. Thus, subjects are required with known conditions to facilitate the machine learning process and such subjects are identified herein as reference subjects.

After completing the production of the apparatus, including the downloading of data at step 2102, actual deployment, invoking the method identified above, is identified by steps 2103. During deployment steps 2103, the condition of each subject is not known and as used herein, these subjects are identified as test subjects.

As is known to those skilled in the art, commercial systems are available for implementing learning procedures resulting in the generation of data that may be deployed within the operational environment 2103.

Referring to the machine learning procedures 2101, a first reference subject is examined at step 2111, using techniques substantially similar to those deployed during the 2103 procedures and detailed with reference to FIG. 23 and FIG. 24. Thus, the examination procedure at step 2111 produces output data consisting of monitored output signals and temperature measurements.

The condition of the reference subject is known, which allows the machine learning process to be taught at step 2112. This in turn allows reference data to be recorded at step 2113 whereafter, at step 2114, a question is asked as to whether another reference subject is to be considered. Thus, when answered in the affirmative, the next reference subject is examined at step 2111, allowing further machine learning to be conducted, such that the reference data is refined over a period of time.

Experiments have shown that, in this particular environment, the recorded reference data does converge towards a workable solution such that, given new input data consisting of the monitored data and the temperature data, it is possible to make an accurate evaluation of the subject's condition.

In the deployment stages 2103, a test subject is selected at step 2121. In a clinical environment the apparatus shown in FIG. 1 may, for example, be deployed for many individual subjects, with appropriate sterilisation procedures being performed between deployments. Thus, in an embodiment, it is possible to replace the dielectric membrane 106 between deployments. For the apparatus shown in FIG. 11 and for the apparatus shown in FIG. 13, it is likely that only a single subject will make use of the apparatus although, over a period of time, many deployments may take place.

At step 2122, the examination is performed and output results are displayed at step 2123. A question is then asked at step 2124 as to whether another subject is to be examined and when answered in the affirmative, the next subject is selected at step 2121.

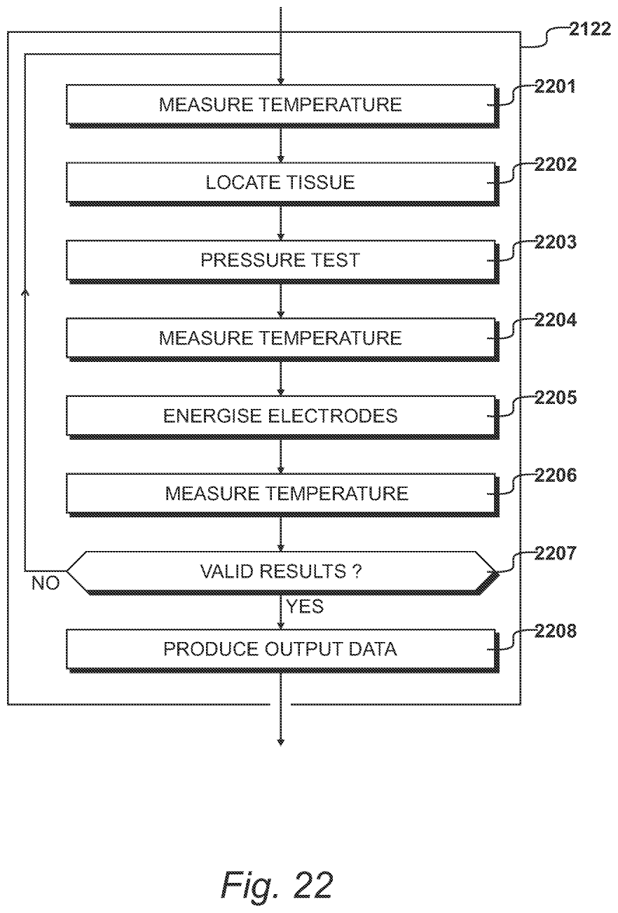

### FIG. 22

An example of process 2122 for performing an examination is shown in FIG. 22. After initializing the apparatus, a temperature measurement is made at step 2201. Thus, this first temperature measurement effectively records the ambient temperature prior to performing the examination.

At step 2202, tissue is located which, for the apparatus shown in FIG. 1 and FIG. 11, involves locating a finger upon the insulated electrodes mounted on the flexible electric membrane. For the apparatus described with reference to FIG. 13, this locating step involves wearing the apparatus.

For the first embodiment and as described with reference to FIG. 5 to FIG. 8, a pressure test is performed and, as previously described, a subject is invited to press harder if the pressure applied is considered to be insufficient.

Upon successfully passing the pressure test at step 2203, a temperature measurement is made at step 2204. Due to the tissue being located, the temperature measured at step 2204 should be higher than the temperature measured at step 2201.

After the temperature measurement at step 2204, the electrodes are energized at step 2205 and data is obtained. An example of these energizing operations will be described with reference to FIG. 23 and FIG. 24.

After performing an energizing cycle, temperature is again measured at step 2206. A question is then asked at step 2207 as to whether valid results have been obtained. The data obtained should be consistent with the reference data recorded at step 2113 and an error message may be generated should this not be the case. At step 2207 it is also possible for the temperature measured at step 2204 to be compared with the temperature measured at step 2206. If a large discrepancy exists (greater than, say, two degrees Celsius) significant additional warming will have occurred during the energization step 2205, therefore the results may be treated again as not being valid; resulting in the question asked at step 2207 being answered in the negative and the process being repeated.

If the temperature measured at step 2206 is substantially the same as the temperature measured at step 2204 and the monitored data is also considered to be valid, the question asked at step 2207 is answered in the affirmative and output data is produced. In the embodiment, if a small difference exists between the temperature measured at step 2204 and the temperature measured at step 2206, the two values may be averaged and this average value is then used to produce output data.

### FIG. 23

An example of procedure 2205 for energizing electrodes is illustrated in FIG. 23. In this embodiment, the processor 402 is configured to select a first set of n electrodes from a set of substantially parallel electrodes. The parallel electrodes may be linear, as shown in FIG. 2 or may be circular as shown in FIG. 19. Capacitively coupled electrode pairs are established, in which each of the first set of n electrodes is capacitively coupled with a second set of m electrodes. Each set of m electrodes are the nearest neighbouring electrodes to an electrode selected from the first set of n electrodes and the number of electrodes present in the second set of m electrodes represents a degree of layering.

As used herein, layering refers to the depth of penetration within the tissue. With greater spacing between electrodes, a greater degree of penetration is achieved. Thus, by performing multiple energizations with differing spacings between electrodes, it is possible to obtain results representing characteristics at different layers or depths.

In the example shown in FIG. 23, a total of fifteen parallel electrodes are present, identified as electrode 2301 to electrode 2315, shown in cross-section. A first set of n electrodes is selected which can consist of any number of electrodes from one to fifteen, but with the range of n electrodes being contiguous with no gaps. For the purposes of this example, n is equal to 2 such that, in this example, the first electrode 2301 will be capacitively coupled with a second set of m electrodes, followed by the second electrode 2302 being capacitively coupled with a second set of m electrodes. In this example, m is equal to 7, which in turn results in the degree of layering being equal to 7.

On the iteration shown in FIG. 23, the first electrode 2301 is identified as an electrode in common. It is sequentially coupled with the second set, consisting of the second electrode 2302 to the eighth electrode 2308. The actual ordering in which coupling occurs is not relevant but a sequential ordering facilitates the creation of program instructions for the processor 402. Thus, in this example, the first electrode 2301 is energized and the second electrode 2302 is monitored, resulting in electric field 2321. The first electrode 2301 continues as the electrode in common but on the next iteration, the third electrode 2303 is monitored, resulting in the generation of electric field 2322. As illustrated in FIG. 23, electric field 2322 penetrates further into the tissue compared to electric field 2321.

On the next iteration, the first electrode 2301 remains the electrode in common and the fourth electrode 2304 is monitored, resulting in the generation of a third electric field 2303. Thus, this process continues until all m electrodes, up to the eighth electrode 2308, have been monitored.

### FIG. 24

As previously described, the first set of n electrodes consisted of two electrodes 2301 and 2302, such that the first electrode 2301 is established as an electrode in common, as described with reference to FIG. 23, followed by the second electrode 2302 being selected as the electrode in common, as shown in FIG. 24. Again, the second set of m electrodes comprises seven electrodes in this example, such that capacitive coupling will occur between the second electrode 2302 and the third electrode 2303, followed by coupling between the second electrode 2302 and the fourth electrode 2304, followed by coupling between the second electrode 2302 and the fifth electrode 2305 and so on, until coupling has occurred between electrode in common 2302 and the ninth electrode 2309.

After performing the operations described with reference to FIG. 23 and FIG. 24, the resulting data is held until a further temperature measurement is taken at step 2206. If the results are valid, output data is produced at step 2208. This output data may reassure a subject or may inform a subject to the effect that a further medical intervention or examination is required.

The invention claimed is:

1. An apparatus for performing non-invasive medical examinations in response to generated electric fields, comprising:
a plurality of insulated electrodes mounted on a flexible dielectric membrane;
a dielectric spacer having a first surface in contact with said flexible dielectric membrane, a second surface, and a window between said first surface and said second surface; and
an infra-red sensor located on said second surface and configured to receive infra-red radiation from said flexible dielectric membrane via said window to determine a temperature of said flexible dielectric membrane.

2. The apparatus of claim 1, wherein sides of said window defined by said dielectric spacer are angled to present a wider opening on said first surface, at a position of said flexible dielectric membrane, compared to said second surface at a position of said infra-red sensor.

3. The apparatus of claim 1, wherein:
said non-invasive medical examinations detect a concentration of one or more chemicals within circulating blood;
said plurality of insulated electrodes are configured to be contacted by a finger; and
said plurality of insulated electrodes are substantially linear and substantially parallel.

4. The apparatus of claim 3, comprising additional insulated electrodes, wherein said additional insulated electrodes are:
substantially linear and parallel;
mounted on opposite side of said flexible dielectric membrane; and
substantially orthogonal to said plurality of insulated electrodes.

5. The apparatus of claim 3, further comprising a processor, wherein said processor is configured to:
select a first set of n electrodes from said plurality of insulated electrodes; and
establish capacitively coupled electrode pairs, in which each of said first set of n electrodes is capacitively coupled with a second set of m electrodes from said plurality of insulated electrodes, wherein
each said second set of m electrodes are a nearest neighbouring electrodes to an electrode selected from said first set of n electrodes; and
a number of electrodes present in said second set of m electrodes represents a degree of layering.

6. The apparatus of claim 4, wherein said dielectric spacer comprises a raised portion arranged to extend into an opening within an upper circuit board to support said flexible dielectric membrane.

7. The apparatus of claim 5, wherein:
said infra-red sensor is located on an intermediate circuit board;
said intermediate circuit board is in contact with a force sensor; and
said processor is configured to inhibit examination procedures when an applied force is below a predetermined threshold.

8. The apparatus of claim 1, wherein:
said non-invasive medical examinations detect anomalies in breast tissue;
said flexible dielectric membrane is substantially dome-shaped, defining an internal surface arrange to be in contact with a human breast;
said plurality of insulated electrodes comprise a first set of circular electrodes arranged in a configuration of concentric rings; and further comprising
a second set of substantially radial electrodes overlapping said concentric rings.

9. The apparatus of claim 8, further comprising an outer membrane arranged over said substantially dome-shaped flexible dielectric membrane.

10. The apparatus of claim 9, wherein said dielectric spacer is positioned between said outer membrane and said substantially dome-shaped flexible dielectric membrane.

11. A method of performing non-invasive medical examinations, in response to generated electric fields, comprising the steps of:
locating human tissue in contact with a plurality of insulated electrodes mounted on a flexible dielectric membrane; and
selecting a transmitting electrode and a monitoring electrode from said plurality of insulated electrodes, such that electric fields penetrate said human tissue, wherein:
a first surface of a dielectric spacer is in contact with said flexible dielectric membrane;
a window is provided between said first surface and a second surface of said dielectric spacer;
an infra-red sensor is located on said second surface and is configured to receive infra-red radiation from said flexible dielectric membrane via said window, to determine a temperature of said flexible dielectric membrane, and further comprising the steps of:
producing output signals derived from said monitoring electrode; and
compensating said output signals with reference to said determined temperature.

12. The method of claim 11, further comprising the step of angling sides of said window defined by said dielectric spacer to present a wider opening on said first surface, at a position of said flexible dielectric membrane, compared to said second surface at a position of said infra-red sensor.

13. The method of claim 11, wherein:
said step of locating human tissue comprises locating a finger in contact with said plurality of insulated electrodes;
said non-invasive medical examinations detect a concentration of one or more chemicals within circulating blood; and
said plurality of insulated electrodes are substantially linear and substantially parallel.

14. The method of claim 13, wherein said dielectric spacer comprises a raised portion arranged to extend into an opening within an upper circuit board to support said flexible dielectric membrane during said step of locating a finger.

15. The method of claim 11, wherein:
said infra-red sensor is located on an intermediate circuit board;
said intermediate circuit board is in contact with a force sensor; and
a processor is configured to perform a step of inhibiting further operation when an applied force is below a predetermined threshold.

16. The method of claim 11, wherein:
said step of locating human tissue comprises locating breast tissue and said non-invasive medical examinations detect anomalies in said breast tissue, wherein:
said flexible dielectric membrane is substantially dome-shaped, defining an internal surface arranged to be in contact with a human breast;
said plurality of insulated electrodes comprise a first set of circular electrodes arranged in a configuration of concentric rings; and
a second set of substantially radial electrodes overlaps said concentric rings.

17. The method of claim 16, wherein:
an outer membrane is arranged over said substantially dome-shaped flexible dielectric membrane; and
said dielectric spacer is positioned between said outer membrane and said substantially dome-shaped flexible dielectric membrane.

18. The method of claim 17, wherein a plurality of infra-red sensors are positioned between said outer membrane and said substantially dome-shaped flexible dielectric membrane.

19. The method of claim 11, further comprising the step of:
developing instructions and reference data for a processor to facilitate said step of producing output signals by a process of machine learning.

20. The method of claim 19, wherein said process of machine learning comprises the steps of evaluating many examinations in which tissue characteristics are known and a temperature of an evaluating membrane is also known, from which said reference data is developed.
